(19) United States
(12) Patent Application Publication
(10) Pub. No.: US 2003/0171804 A1
(43) Pub. Date: Sep. 11, 2003

(54) STENTS COATED WITH FLUOROALKYL GROUPS

(76) Inventor: WERNER KRAUSE, BERLIN (DE)

Correspondence Address: MILLEN WHITE ZELANO & BRANIGAN, ARLINGTON COURTHOUSE PLAZA 1, 2200 CLARENDON BOULEVARD, SUITE 1400, ARLINGTON, VA, 22201

(21) Appl. No.: 09/446,396

(22) PCT Filed: Jun. 18, 1998
(86) PCT No.: PCT/EP98/03627

(30) Foreign Application Priority Data
Jun. 24, 1997 (DE) 197 27 838.8

(51) Int. Cl. A61F 2/06

(52) U.S. Cl. 623/1.46

(57) ABSTRACT

The invention relates to stents that consist of a stent parent substance that is coated with a carrier polymer to which are connected perfluoroalkyl chains that project from the stent surface like a brush, as well as processes for their production and their use for restenosis prophylaxis.

The invention relates to the field of vascular implants and describes stents that are coated with fluoroalkyl groups, processes for their production and their use for restenosis prophylaxis.

### PRIOR ART

Stents are prior art (Pschyrembel, Klinisches Wörterbuch [Clinical Dictionary] 257th Edition, Verlag W. de Gruyter). Stents are endoprostheses that make it possible to keep open duct-like structures in the bodies of humans or animals (e.g., vascular, esophageal, tracheal and bile duct stents). They are used as palliative measures in the case of stenoses by obstruction (e.g., arteriosclerosis) or external pressure (e.g., in the case of tumors). Radioactive stents are used, for example, after vascular-surgery interventions or radiological interventions (e.g., balloon angioplasty) for restenosis prophylaxis.

There is now the problem that the stent represents a foreign substance for the body, and a reaction of intolerance results.

The object of this invention is therefore to make available stents that are more compatible than conventional stents. This object is achieved by the stents that are described below, as they are characterized in the claims.

### DESCRIPTION OF THE INVENTION

The above-described object is achieved according to the invention in that the surface of the stents is coated with a carrier polymer, from which the fluoroalkyl groups project like a brush.

The device according to the invention thus consists of the parent substance of the stent, which is coated with a carrier polymer to which are connected perfluoroalkyl chains that project from the surface of the stent like a brush.

As a parent substance, the commercially available vascular implants can be used, e.g., a Wiktor stent, a Strecker stent, a Nitinol stent or a Palmaz-Schatz stent. The stent parent substance can be produced in metallic form or from a polymer.

Considered as carrier polymers are, for example, modified polyurethanes, which carry groups that can be derivatized, e.g., amino, hydroxyl, carboxyl, carbonyl, thiol, thiocarboxyl or other groups that can be reacted. The groups that can be derivatized can also be contained in the carrier polymer via polyethylene glycols, polysaccharides, cyclodextrins, polyaminopolycarboxylic acids or proteins.

Polymers that are based on polyamino-p-xylylene (formula I), however, can also be used advantageously as carrier polymers:

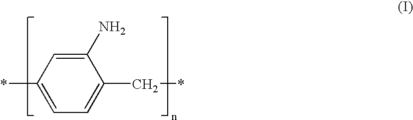

The following polymers can also be used as carrier polymers:

Polyorganosilanes, polyvinylpyrrolidones, polymethylmethacrylates, polyhydroxymethylmethacrylates, mixed polymers from N-vinylpyrrolidone and hydroxymethylmethacrylate, polyamides, polyacrylamides, polyethylenes, polyethylene oxides, polyethylene glycols, polyesters, polypropylene oxides, polysiloxanes, PVC derivatives, polyvinyllactams, polyethylene terephthalates, polysilicones, polysaccharides, proteins, polysulfones or polysulfonates, provided that they contain one or more of the above-mentioned groups that can be derivatized.

Fluoroalkyl chain-containing molecules of general formula II are connected to the carrier polymer:

(X—Y—(CF₂)ₙ—Z)ₘ (II)

In this case,

X means a direct binding to the carrier polymer, an amino, ether, thioether, carbonyl, thiocarbonyl, sulfonyl, sulfuryl or phosphoryl group or a longer bridge, e.g., an alkyl chain, which can be interrupted by heteroatoms and/or can be substituted by heteroatoms, an amino acid, dicarboxylic acid, a peptide, nucleotide or a sugar,

Y means a direct bond, an amino, ether, thioether, carbonyl, thiocarbonyl, sulfonyl, sulfuryl or phosphoryl group or a longer bridge, e.g., an alkyl chain, which can be interrupted by heteroatoms and/or can be substituted by heteroatoms, an amino acid, dicarboxylic acid, a peptide, nucleotide or a sugar,

Z means a fluorine or a hydrogen atom,

n means a natural number that is greater than or equal to 1,

m means a natural number of between 1 and the number of groups that can be derivatized in the carrier polymer.

Preferably n is greater than 5, especially preferably n is greater than 10.

By way of example, the stents according to the invention can be produced as follows:

1. An uncoated stent can first be coated with a carrier polymer (e.g., a polyurethane that can be obtained from the reaction of 3,3′-diacetylamino-diphenylmethane-4,4′-diisocyanate and butanediol and subsequent removal of the protective groups). This polymer is modified in such a way that it carries groups that can be derivatized (amino groups in this example). The polymer is dissolved in a solvent (e.g., chloroform), and the stent is immersed in the polymer solution. After the stent is removed from the polymer solution, it is dried in a drying chamber at room temperature.

2. As an alternative to 1., the carrier polymer can be placed on the stent with the aid of a gas phase deposition or plasma polymerization. This process is based on, e.g., the process that is disclosed in German Laid-Open Specification DE 196 04 173 A1 for the production of antithrombogenic surfaces in medical subjects. In this process, a functionalized polymer is placed on the metallic stent parent substance by gas phase coating at elevated temperatures and reduced pressures.

3. The stent that is coated according to 1. or 2. is mixed with a solution of the derivatizing agent—as described below.

The derivatization is carried out by reaction of groups XH (in this case, X means a group that can be derivatized, e.g., an amino, hydroxyl or thiol group) of the stent (polymer-XH) that is coated with the carrier polymer with compounds of general formula III

Nu—CO—L—R^{F} (III)

in which

R^{F} represents a fluoroalkyl chain,

L can be a direct bond, an alkyl group, which can be interrupted and/or substituted by heteroatoms, an amino acid, a peptide, a nucleotide or a sugar,

Nu means a nucleofuge.

If radicals L contain hydroxyl groups, they can optionally be protected by acetyl or isopropylidene groups. The protective group technique is well-known to one skilled in the art.

As nucleofuges, the radicals

I, Br, Cl, F, —OTs, —OMs,

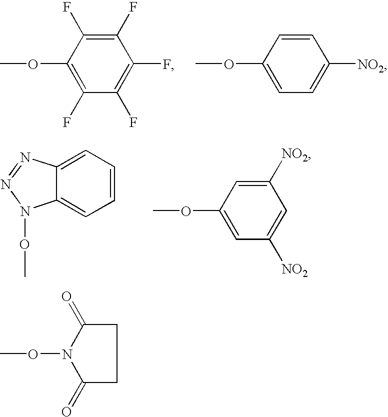

are advantageously used.

The reaction is performed in a mixture of water and organic solvents, such as isopropanol, ethanol, methanol, butanol, dixoane, tetrahydrofuran, dimethylformamide, dimethylacetamide, formamide or dichloromethane. Preferred are ternary mixtures that consist of water, isopropanol and dichloromethane.

The reaction is performed at a temperature range of between −10° C. to 100° C., preferably between 0° C. to 30° C.

As acid traps, inorganic and organic bases such as triethylamine, pyridine, N-methylmorpholine, diisopropylethylamine, dimethylaminopyridine, alkali and alkaline-earth hydroxides, their carbonates or bicarbonates such as lithium hydroxide, sodium hydroxide, potassium hydroxide, sodium carbonate, sodium bicarbonate, potassium bicarbonate are used.

The compounds of general formula III are obtained from compounds of general formula IV

HO₂—C—L—R^{F} (IV),

in which

R^{F} and I have the above-mentioned meaning, according to the processes of acid activation that are generally known to one skilled in the art, such as are carried out by reaction of the acid with dicyclohexylcarbodiimide, N-hydroxysuccinimide/dicyclohexylcarbodiimide, carbonyldiimidazole, 2-ethoxy-1-ethoxy-carbonyl-1,2-dihydroquinoline, oxalic acid dichloride or isobutyl chloroformate in the way that is described in the literature:

Aktivierung von Carbonsauren [Activation of Carboxylic Acids]. Ubersicht in Houben-Weyl [Survey in Houben-Weyl], Methoden der Organischen Chemie [Methods of Organic Chemistry], Volume XV/2, Georg Thieme Verlag Stuttgart, 19

Aktivierung mit Carbodiimiden [Activation with Carbodiimides], R. Schwyzer and H. Kappeler, Helv. 46, 1550 (1963)

E. Wunsch et al., Volume 100, 173 (1967)

Aktivierung mit Carbodiimiden/Hydroxysuccinimid [Activation with Carbodiimides/Hydroxysuccinimide]. J. Am. Chem. Soc. 86: 1839 (1964) and J. Org. Chem. 53: 3583 (1988). Synthesis 453 (1972)

Anhydridmethode, 2-Ethoxy-1-ethoxycarbonyl-1,2-dihydrochinolin [Anhydride Methods, 2-Ethoxy-1-ethoxycarbonyl-1,2-dihydroquinoline]: B. Belleau et al., J. Am. Chem. Soc., 90: 1651 (1986), H. Kunz et al., Int. J. Pept. Prot. Res., 26: 493 (1985) and J. R. Voughn, Am. Soc. 73: 3547 (1951)

Imidazolid-Methode [Imidazolide Method]: B. F. Gisin, R. B. Menifield, D. C. Tosteon, Am. Soc 91: 2691 (1969)

Saurechlorid-Methoden, Thionylchlorid [Acid Chloride Methods, Thionyl Chloride]: Helv., 42: 1653 (1959)

Oxalylchlorid [Oxalyl Chloride], J. Org. Chem., 29: 843 (1964).

The compounds of general formula IV are commercially available products (Fluorochem, ABCR) or are obtained from compounds of general formula V

H—Q—L—R^{F} (V)

with

Q in the meaning of

—O— or —S— or

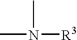

or

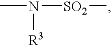

with a binding of the nitrogen atom to the hydrogen atom or

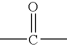

by reaction with compounds of general formula VI

Hal—CH₂—CO—OR¹ (VI)

with

Hal in the meaning of Cl, Br, I and

R¹ in the meaning of H, methyl, ethyl, t-butyl, benzyl, isopropyl, represented, for example, according to C. F. Ward, Soc. 121, 1161 (1922), according to the methods that are known to one skilled in the art, such as alkylation of alcohols with alkyl halides [Houben-Weyl, Methoden der Organischen Chemie, Sauerstoffverbindungen [Oxygen Compounds], Part 3, Methoden zur Herstellung und Umwandlung von Ethern [Methods for the Production and Conversion of Ethers], Georg Thieme Verlag, Stuttgart 1965, Alkylierung von Alkoholen mit Alkylhalogeniden [Alkylation of Alcohols with Alkyl Halides], p. 24, Alkylierung von Alkoholen mit Alkylsulfaten [Alkylation of Alcohols with Alkylsulfates] p. 33] or N-Alkylierung eines Sulfonamids mit Alkylsulfonaten [N-Alkylation of a Sulfonamide with Alkylsulfonates] [Houben-Weyl, Methoden der organischen Chemie, XI/2 Stickstoffverbindungen [Nitrogen Compounds], Georg Thieme Stuttgart, 1957, p. 680, J. E. Rickman and T. Atkins, Am. Chem. Soc., 96: 2268, 1974, 96: 2268; F. Chavez and A. D. Sherry, J. Org. Chem. 1989, 54: 2990].

For the case that Q means group

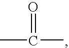

the reaction is performed with a Wittig-reagent of the structure

+−

(AR)₃P—CH—(CH₂)ᵣ—CO₂R⁴,

whereby r means numbers 0-16. The CH═CH double bond that is produced is this case can be maintained as a component of the structure or can be converted into a —CH₂—CH₂ grouping by catalytic hydrogenation (Pd 5%/C).

The compounds of general formula VI are commercially available products (Fluorochem, ABCR).

The above-described processes are generally performed at temperatures of 0-80° C. When the stent is coated with the polymer, solvents can be used depending on the respective polymer. When a non-aqueous solvent is used, the latter is to be removed before the implantation.

The derivatization of groups XH (in this case X means a carboxyl group) of polymer-coated stents (polymer-COOH) is carried out analogously with compounds of general formula VII

H₂N—L—R^{F} (VII)

by activation of the COOH groups of the polymer as described above. In this case, L and R^{F} have the above-described meaning.

The operations that are necessary for implementing the above process that is described in principle are known to one skilled in the art. Special embodiments are described in detail in the examples.

The stents according to the invention achieve the above-described object. The stents according to the invention are physiologically well-tolerated.

The surface coating of the stents according to the invention, as it was described above, can also be used in general in the coating of surfaces to make the latter inert. This in particular for medical applications, e.g., for catheters, probes, dialyzers, artificial cardiac valves, protheses, etc.

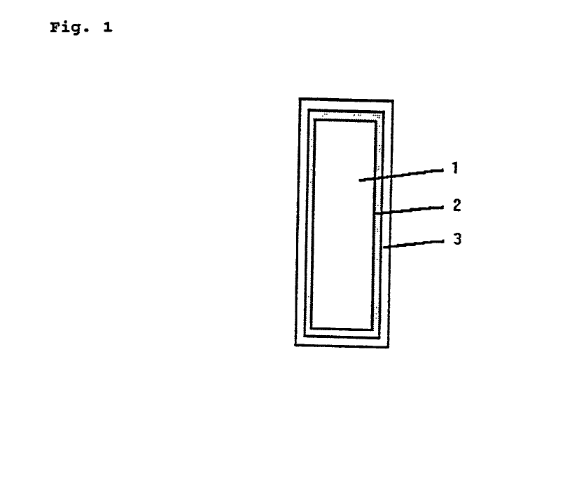

FIG. 1 shows diagrammatically the structure of the stents according to the invention. Here,

1 means stent parent substance

2 means carrier polymer

3 means the fluoroalkyl-chain-carrying layer.

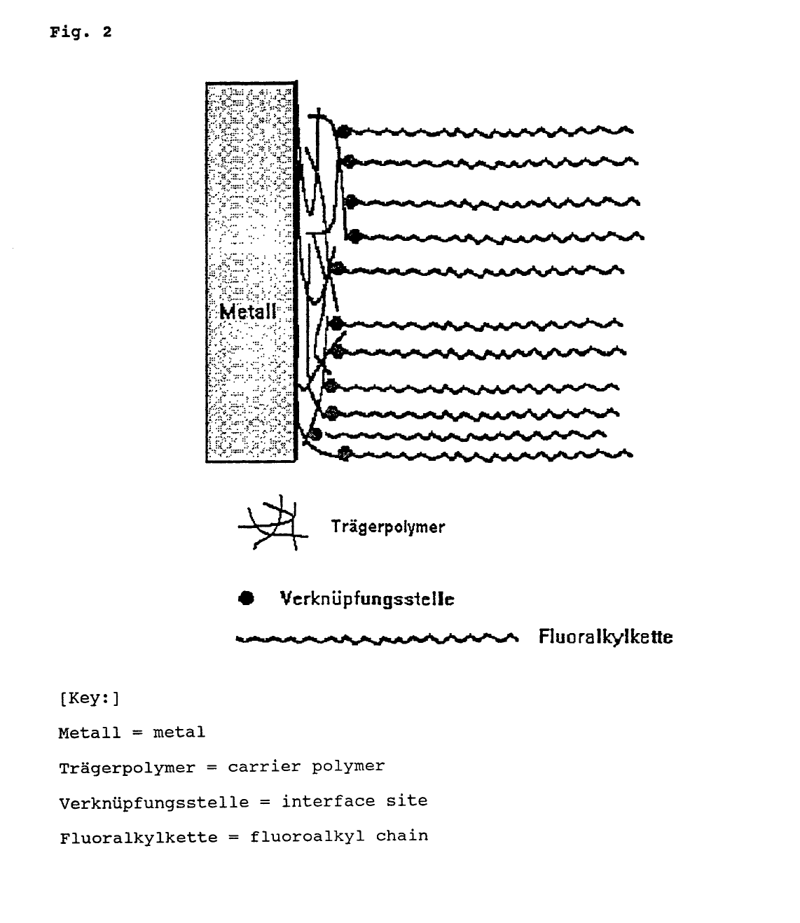

FIG. 2 is a visualization in which the brush-like structure of the fluoroalkyl-chain-carrying layer is shown diagrammatically.

Embodiments:

The following examples are to explain the subject of the invention, without intending that it be limited to these examples.

### EXAMPLE 1

Polyurethane, which can be obtained by reaction of 3,3′-diacetylamino-diphenylmethane-4,4′-diisocyanate and butanediol, is used as a carrier polymer. After the polymerization, the acetyl protective groups are removed. The stents are coated in that they are immersed in a 5% chloroform solution of the polymer. Then, they are allowed to dry in a clean-room-drying chamber at room temperature. The average layer thickness is 20 μm. The derivatization with fluoroalkyl groups is carried out by reaction of free amino groups with the acid chloride of formula VIII

Cl—CO—(CF₂)₁₄—CF₃ (VIII),

as it is described in the literature and is familiar to one skilled in the art. After the drying, the stent is ready for use.

### EXAMPLE 2

The coating of the stent with a polymer, which carries free carboxyl groups on the surface, is carried out as described under Example 1. Then, the reaction with thionyl chloride is carried out in an acid chloride, as is known to one skilled in the art. Then, the chlorine atoms of the acid chloride are reacted with an amine of formula IX

H₂N—(CF₂)₁₆—CF₃ (IX)

After the drying, the stent is ready for use.

### EXAMPLE 3

The coating of a metal stent by CVD-polymerization (CVD Chemical Vapor Deposition) of 4-amino-[2,2]-paracyclophane is carried out in a suitably designed unit. The unit is connected to an argon cylinder, since argon acts as a carrier gas. The argon feeder is connected to a 380 mm quartz glass pipe that has an outside diameter of 30 mm. The quartz glass pipe is connected on its other end to a high-grade steel vessel. The quartz glass pipe is stored freely suspended in a three-zone tubular pipe furnace, which has a heated length of 320 mm and an inside diameter of 32 mm. All three heating zones can be heated to 800° C. The stent that is to be coated is attached to the sample holder via the removable inspection glass. Then, the reactor is sealed again, and the unit is put into operation by actuating the main switch. At the same time, the two cooling circuits are activated, and the vessel wall is heated to 100° C. Then, a porcelain boat with a weighted amount of monomer is placed in the sublimation zone, and the latter is sealed again. The reactor is then pumped off to a basic pressure of 0.03 mbar. A carrier gas stream of 20 sccm is now set, and then a working pressure of 0.2 mbar is imposed. A waiting period is now carried out until both the carrier gas flow and the working pressure are constant. The desired pyrolysis temperature of 680° C. is now imposed, and a waiting period is carried out until this temperature is reached in the pyrolysis zone. Then, the sample holder can be rotated with a rotating speed of 20 rpm, and the sublimation zone is heated to 290° C. The coating process is verified with the aid of the layer thickness monitor. When the desired layer thickness of 280 nm is reached, the coating process can be ended. Here, the furnace controller, the rotary motor of the sample holder and the carrier gas stream are stopped, the butterfly valve is opened, and once more pumped off to basic pressure. Then, the pump is turned off, the unit is aerated via the aeration valve, and the sample is removed.

Derivatization with fluoroalkyl groups is carried out as in Example 1 by reaction of the free amino groups on the carrier polymer with the acid chloride of formula VIII

Cl—CO—(CF₂)₁₄—CF₃ (VIII),

as is described in the literature and as is familiar to one skilled in the art. After drying, the stent is ready for use.

1. Stent, characterized in that it consists of a stent parent substance that is coated with a carrier polymer to which are connected the perfluoroalkyl chains that project from the stent surface like a brush.

2. Stent according to claim 1, wherein the stent parent substance is a metallic stent parent substance or a stent that is produced from a polymer.

3. Stent according to claim 2, wherein the metallic parent substance is a Wiktor stent, a Palmaz-Schatz stent, a Strecker stent, or a Nitinol stent.

4. Stent according to claim 1, wherein the carrier polymer is one of the following polymers: a polyurethane derivative, a polyamino-p-xylylene derivative, a polyorganosilane, a polyvinylpyrrolidone, a polymethylmethacrylate, a polyhydroxymethylmethacrylate, a mixed polymer from N-vinylpyrrolidone and hydroxymethylmethacrylate, a polyamide, a polyacrylamide, a polyethylene, a polyethylene oxide, a polyethylene glycol, a polyester, a polypropylene oxide, a polysiloxane, a PVC derivative, a polyvinyllactam, a polyethylene terephthalate, a polysilicone, a polysaccharide, a protein, a polysulfone or a polysulfonate.

5. Stent according to claim 1, wherein fluoroalkyl-chain-containing molecules of general formula II are connected to the carrier polymer
(X—Y—(CF₂)ₙ—Z)ₘ (II)
in which
X stands for a direct binding to the carrier polymer, an amino, ether, thioether, carbonyl, thiocarbonyl, sulfonyl, sulfuryl or phosphoryl group or a longer bridge to the carrier polymer, e.g., an alkyl chain, which can be interrupted by heteroatoms and/or can be substituted by heteroatoms, an amino acid, a dicarboxylic acid, a peptide, nucleotide or a sugar,
Y stands for a direct bond, an amino, ether, thioether, carbonyl, thiocarbonyl, sulfonyl, sulfuryl or phosphoryl group or a longer bridge such as, e.g., an alkyl chain, which can be interrupted by heteroatoms and/or can be substituted by heteroatoms, an amino acid, a dicarboxylic acid, a peptide, nucleotide or a sugar,
Z stands for a fluorine atom or a hydrogen atom,
n stands for a natural number that is greater than or equal to 1,
m stands for a natural number of between 1 and the number of groups that can be derivatized in the carrier polymer.

6. Process for the production of a stent according to one of the preceding claims, wherein a stent parent substance is coated with a carrier polymer, and then the surface is derivatized with perfluoroalkyl-chain-containing molecules.

7. Process according to claim 6, wherein the carrier polymer is placed on the stent parent substance by gas phase coating or plasma polymerization.

8. Surface coating, wherein perfluoroalkyl chains that project from the surface like a brush are connected to a carrier polymer.

9. Surface coating according to claim 8, wherein fluoroalkyl-chain-containing molecules of general formula II are connected to the carrier polymer:
(X—Y—(CF₂)_{n—Z)}ₘ (II)
in which
X stands for a direct binding to the carrier polymer, an amino, ether, thioether, carbonyl, thiocarbonyl, sulfonyl, sulfuryl or phosphoryl group or a longer bridge to the carrier polymer, such as, e.g., an alkyl chain, which can be interrupted by heteroatoms and/or can be substituted by heteroatoms, an amino acid, a dicarboxylic acid, a peptide, nucleotide or a sugar,
Y stands for a direct bond, an amino, ether, thioether, carbonyl, thiocarbonyl, sulfonyl, sulfuryl or phosphoryl group or a longer bridge, such as, e.g., an alkyl chain, which can be interrupted by heteroatoms and/or can be substituted by heteroatoms, an amino acid, a dicarboxylic acid, a peptide, nucleotide or a sugar,
Z stands for a fluorine or a hydrogen atom,
n stands for a natural number that is greater than or equal to 1,
m stands for a natural number of between 1 and the number of groups that can be derivatized in the carrier polymer.
